(19) United States
(12) Patent Application Publication
(10) Pub. No.: US 2025/0122158 A1
(43) Pub. Date: Apr. 17, 2025

(54) PROCESS FOR PREPARING ISOXAZOLINE-5,5-VINYLCARBOXYLIC ACID DERIVATIVES

(71) Applicant: BAYER AKTIENGESELLSCHAFT, Leverkusen (DE)

(72) Inventors: Anton LISHCHYNSKYI, Leverkusen (DE); Frank MEMMEL, Leverkusen (DE); Mark James FORD, Leverkusen (DE); Wahed Ahmed MORADI, Leverkusen (DE)

(21) Appl. No.: 18/715,254

(22) PCT Filed: Dec. 1, 2022
(86) PCT No.: PCT/EP2022/083993

§ 371 Date: May 31, 2024

(30) Foreign Application Priority Data
Dec. 3, 2021 (EP) 21212349.1

(51) Int. Cl. C07D 261/04

(52) U.S. Cl. CPC C07D 261/04

(57) ABSTRACT

The present invention relates to a novel process for preparing isoxazoline-5,5-vinylcarboxylic acid derivatives of the formula (I)

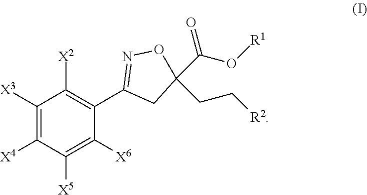

The present invention relates to a novel process for preparing isoxazoline-5,5-vinylcarboxylic acid derivatives of the formula (I), isoxazoline-5,5-vinylcarboxylic acid derivatives of the formula (I), and to the intermediate compounds of the formulae (II) and (IV) that occur in the process.

Isoxazoline-5,5-vinylcarboxylic acid derivatives of the general formula (I) are important precursors of active agrochemical ingredients (cf. WO2018/228985).

WO2018/228985 already describes a process for preparing isoxazoline-5,5-vinylcarboxylic acid derivatives of the general formula (I). However, the process described therein is unsuitable for an industrial scale synthesis on account of the use of reactants that are unavailable on an industrial scale, for example trifluoromethanesulfonic anhydride or the base diazabicycloundecene (DBU).

It was thus an object of the invention to provide a process for preparing isoxazoline-5,5-vinylcarboxylic acid derivatives of the general formula (I), which is suitable for synthesis on an industrial scale, and nevertheless has a high yield, such that laborious purification methods can be dispensed with.

The object was achieved in accordance with the invention by a process for preparing isoxazoline-5,5-vinylcarboxylic acid derivatives of the general formula (I)

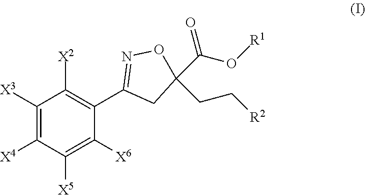

in which
X² is H, C₁-C₄-alkyl, C₁-C₄-fluoroalkyl, C₁-C₄-fluoroalkoxy, C₁-C₄-alkoxy, fluorine or CN,
X³ is H, C₁-C₄ alkyl, C₁-C₄ fluoroalkyl, C₁-C₄ fluoroalkoxy, C₁-C₄ alkoxy, fluorine, chlorine or CN,
X⁴ is H, C₁-C₄-alkyl, C₁-C₄-fluoroalkyl, C₁-C₄-fluoroalkoxy, C₁-C₄-alkoxy, fluorine or CN,
X⁵ is H, C₁-C₄ alkyl, C₁-C₄ fluoroalkyl, C₁-C₄ fluoroalkoxy, C₁-C₄ alkoxy, fluorine, chlorine or CN,
X⁶ is H, C₁-C₄-alkyl, C₁-C₄-fluoroalkyl, C₁-C₄-fluoroalkoxy, C₁-C₄-alkoxy, fluorine or CN,
R¹ is branched C₃-C₈-alkyl, n-C₃-C₈-alkyl, C₃-C₈-cycloalkyl, unsubstituted benzyl, unsubstituted phenyl or mono- or di-C₁-C₃-alkyl-substituted benzyl or phenyl and
R² is H or C₁-C₃-alkyl,
characterized in that compounds of general formula (II)

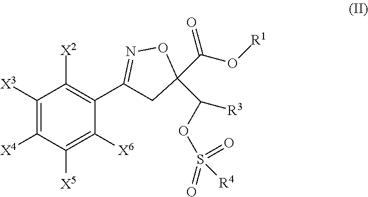

in which R¹ and X² to X⁶ have the meanings given above,
R³ is C₁-C₄-alkyl and
R⁴ is C₁-C₄-alkyl, unsubstituted phenyl or mono- or di-C1-C3-alkyl-substituted phenyl;
are heated to temperatures of 100 to 240° C. in the presence of a base. (Step 1)

By the process according to the invention, the compounds of the formula (I) are obtained in high yields, preferably of more than 75%. There is likewise no need to use any compounds that are unavailable on an industrial scale.

When leaving groups other than the —SO₂CF₃ group described in the prior art are used in compounds of the formula (II), elimination to give the vinyl group achieves only a poor yield as a result of formation of secondary components. It has been found that, surprisingly, especially through suitable selection of the variable R¹, it was possible to distinctly increase the yield of the process according to the invention, even in the case of use of nonfluorinated leaving groups, for example —SO₂CH₃, and to reduce the formation of unwanted secondary components.

In a particular configuration of the invention, the process according to the invention further encompasses the preparation of the compounds of the formula (II)

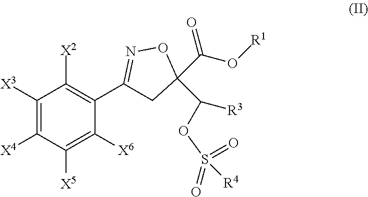

by reaction of compounds of the formula (IV)

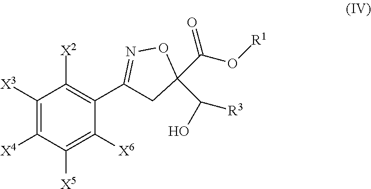

in which R¹, R³, R⁴ and X² to X⁶ have the meanings given above with R⁴SO₂Cl or (R⁴SO₂)₂O, where R⁴ has the meaning given above,
in the presence of a base. (Step 0-2)

In a further particular configuration of the invention, the process according to the invention further comprises the preparation of the compounds of the formula (IV) by reaction of compounds of the formula (III)

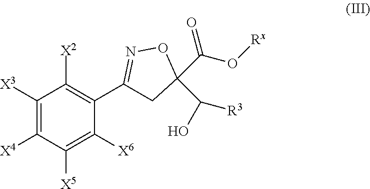

in which
R³ and X² to X⁶ have the meanings given above and
R^{x} is H or C₁-C₃-n-alkyl, where, if R¹ is n-propyl, R^{x} is not n-propyl,
with compounds of the formula R¹—OH in which R¹ has the definition given above. (Step 0-1)

In a particularly preferred configuration of the invention, the compounds of the formula (I)

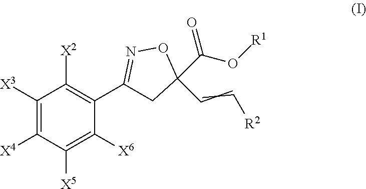

in which R¹, R² and X² to X⁶ have the definitions given above
are also hydrolysed in the presence of a base and then protonated in the presence of an acid or alternatively hydrolysed in the presence of an acid to give compounds of the formula (V)

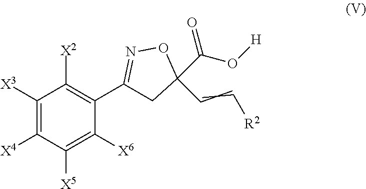

in which R² and X² to X⁶ have the meanings given above. (Step 2)

The invention further provides the compounds of the formula (I)

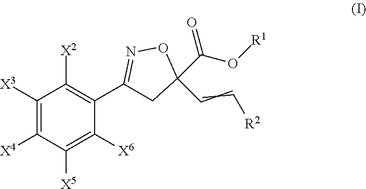

in which R¹, R² and X² to X⁶ have the definitions given above.

Especially preferred here is the compound isopropyl 3-(3,5-difluorophenyl)-5-vinyl-4H-isoxazole-5-carboxylate.

The invention further provides the compounds of the formula (II)

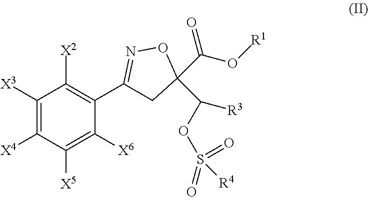

in which R¹, R³, R⁴ and X² to X⁶ have the meanings given above.

Especially preferred here is the compound isopropyl 3-(3,5-difluorophenyl)-5-(1-methylsulfonyloxyethyl)-4H-isoxazole-5-carboxylate.

The invention further provides the compounds of the formula (IV)

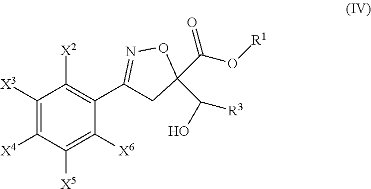

in which R¹, R³ and X² to X⁶ have the definitions given above.

Especially preferred here is the compound isopropyl 3-(3,5-difluorophenyl)-5-(1-hydroxyethyl)-4H-isoxazole-5-carboxylate.

The preferred embodiments described below refer, if appropriate, to all formulae described herein.

Preferred radical definitions for X² to X⁶ are as follows:
X² is H, methyl, trifluoromethyl, difluoromethyl, difluoromethoxy, trifluoromethoxy, fluorine, methoxy or CN,
X³ is H, methyl, trifluoromethyl, difluoromethyl, difluoromethoxy, trifluoromethoxy, fluorine, chlorine, methoxy or CN,
X⁴ is H, methyl, trifluoromethyl, difluoromethyl, difluoromethoxy, trifluoromethoxy, fluorine, methoxy or CN,
X⁵ is H, methyl, trifluoromethyl, difluoromethyl, difluoromethoxy, trifluoromethoxy, fluorine, chlorine, methoxy or CN,
X⁶ is H, methyl, trifluoromethyl, difluoromethyl, difluoromethoxy, trifluoromethoxy, fluorine, methoxy or CN.

Particularly preferred radical definitions for X² to X⁶ are as follows:
X² is H,
X³ is H, methyl, trifluoromethyl, difluoromethyl, fluorine, chlorine, methoxy or CN,
X⁴ is fluorine, H,
X⁵ is H, methyl, trifluoromethyl, difluoromethyl, fluorine, chlorine, methoxy or CN,
X⁶ is H.

Very particularly preferred radical definitions for X² to X⁶ are as follows:
X² is H,
X³ is H or fluorine,
X⁴ is H or fluorine,
X⁵ is H or fluorine,
X⁶ is H.

Most preferred radical definitions for X² to X⁶ are as follows:
X² is H,
X³ is fluorine,
X⁴ is H,
X⁵ is fluorine,
X⁶ is H.

With regard to the further configurations of the invention:
R¹ is preferably isopropyl, n-propyl, 2-methyl-1-propyl, 1-methyl-1-propyl, cyclohexyl, 3-methyl-1-butyl, 1-butyl, 1-pentyl, benzyl or tert-butyl, more preferably isopropyl, 2-methyl-1-propyl, 1-methyl-1-propyl, cyclohexyl, 3-methyl-1-butyl, 1-butyl or 1-pentyl, even more preferably isopropyl, 2-methyl-1-propyl, 1-methyl-1-propyl, cyclohexyl, 3-methyl-1-butyl, and most preferably isopropyl or 2-methyl-1-propyl.
R² is preferably H, methyl or ethyl, more preferably H or methyl, and even more preferably H.
R³ is preferably methyl, ethyl, isopropyl or n-propyl, more preferably methyl or ethyl, and even more preferably methyl.

R⁴ is preferably C₁-C₄-alkyl or p-tolyl, more preferably C₁-C₂-alkyl or p-tolyl, even more preferably methyl or p-tolyl, and most preferably methyl.

Other preferred radical definitions are as follows:
R¹ is isopropyl, n-propyl, 2-methyl-1-propyl, 1-methyl-1-propyl, cyclohexyl, 3-methyl-1-butyl, 1-butyl, 1-pentyl, benzyl or tert-butyl,
R² is H, methyl or ethyl,
R³ is methyl, ethyl, isopropyl or n-propyl,
R⁴ is C₁-C₄-alkyl or p-tolyl and
R^{x} is H, methyl, ethyl or n-propyl, where, if R¹ is n-propyl, R^{x} is not n-propyl.

Other particularly preferred radical definitions are as follows:
R¹ is isopropyl, 2-methyl-1-propyl, 1-methyl-1-propyl, cyclohexyl, 3-methyl-1-butyl, 1-butyl or 1-pentyl,
R² is H, methyl or ethyl,
R³ is methyl, ethyl, isopropyl or n-propyl,
R⁴ is C₁-C₂-alkyl or p-tolyl and
R^{x} is H, methyl, ethyl or n-propyl.

Other very particularly preferred radical definitions are as follows:
R¹ is isopropyl, 2-methyl-1-propyl, 1-methyl-1-propyl, cyclohexyl, 3-methyl-1-butyl,
R² is H or methyl,
R³ is methyl or ethyl,
R⁴ is methyl or p-tolyl and
R^{x} is H, methyl or ethyl.

Other most preferred radical definitions are as follows:
R¹ is isopropyl or 2-methyl-1-propyl,
R² is H,
R³ is methyl,
R⁴ is methyl and
R^{x} is H or methyl.

Other preferred radical definitions are as follows:
X² is H, methyl, trifluoromethyl, difluoromethyl, difluoromethoxy, trifluoromethoxy, fluorine, methoxy or CN,
X³ is H, methyl, trifluoromethyl, difluoromethyl, difluoromethoxy, trifluoromethoxy, fluorine, chlorine, methoxy or CN,
X⁴ is H, methyl, trifluoromethyl, difluoromethyl, difluoromethoxy, trifluoromethoxy, fluorine, methoxy or CN,
X⁵ is H, methyl, trifluoromethyl, difluoromethyl, difluoromethoxy, trifluoromethoxy, fluorine, chlorine, methoxy or CN,
X⁶ is H, methyl, trifluoromethyl, difluoromethyl, difluoromethoxy, trifluoromethoxy, fluorine, methoxy or CN,
R¹ is isopropyl, n-propyl, 2-methyl-1-propyl, 1-methyl-1-propyl, cyclohexyl, 3-methyl-1-butyl, 1-butyl, 1-pentyl, benzyl or tert-butyl,
R² is H, methyl or ethyl,
R³ is methyl, ethyl, isopropyl or n-propyl,
R⁴ is C₁-C₄-alkyl or p-tolyl and
R^{x} is H, methyl, ethyl or n-propyl, where, if R¹ is n-propyl, R^{x} is not n-propyl.

Other particularly preferred radical definitions are as follows:
X² is H,
X³ is H, methyl, trifluoromethyl, difluoromethyl, fluorine, chlorine, methoxy or CN,
X⁴ is fluorine, H,
X⁵ is H, methyl, trifluoromethyl, difluoromethyl, fluorine, chlorine, methoxy or CN,
X⁶ is H,
R¹ is isopropyl, 2-methyl-1-propyl, 1-methyl-1-propyl, cyclohexyl, 3-methyl-1-butyl, 1-butyl or 1-pentyl,
R² is H, methyl or ethyl,
R³ is methyl, ethyl, isopropyl or n-propyl,
R⁴ is C₁-C₂-alkyl or p-tolyl and
R^{x} is H, methyl, ethyl or n-propyl.

Other very particularly preferred radical definitions are as follows:
X² is H,
X³ is H or fluorine,
X⁴ is H or fluorine,
X⁵ is H or fluorine,
X⁶ is H,
R¹ is isopropyl, 2-methyl-1-propyl, 1-methyl-1-propyl, cyclohexyl, 3-methyl-1-butyl,
R² is H or methyl,
R³ is methyl or ethyl,
R⁴ is methyl or p-tolyl and
R^{x} is H, methyl or ethyl.

Other most preferred radical definitions are as follows:
X² is H,
X³ is fluorine,
X⁴ is H,
X⁵ is fluorine,
X⁶ is H,
R¹ is isopropyl or 2-methyl-1-propyl,
R² is H,
R³ is methyl,
R⁴ is methyl and R^{x} is H or methyl.

The compounds of the formulae (I), (II), (III), (IV) and (V) may take the form of isomer mixtures: The isomer ratio between (Ia) and (Tb), (IIa) and (IIb), (IIIa) and (IIIb), (IVa) and (IVb) and (Va) and (Vb) varies; in general, (Ia), (IIa), (IIIa), (IVa) or (Va) is present in excess.

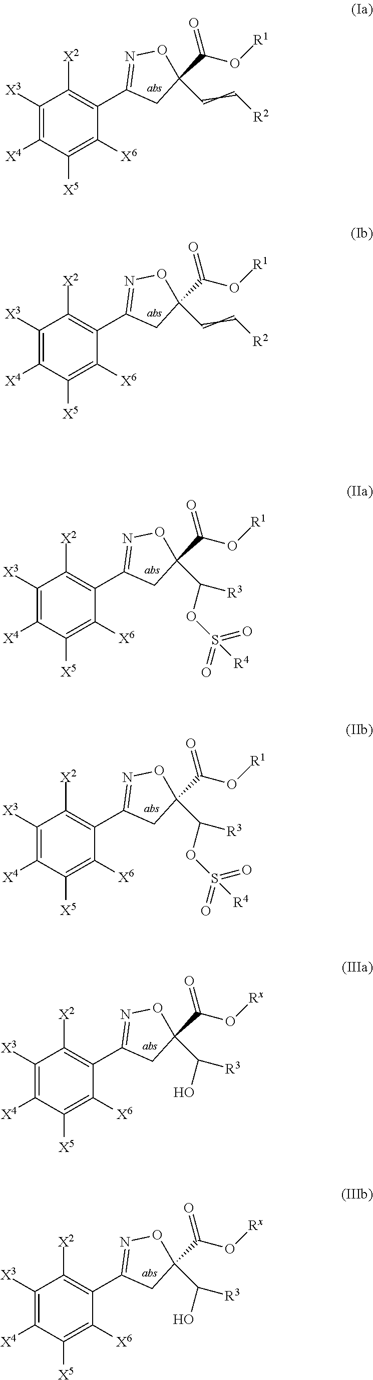

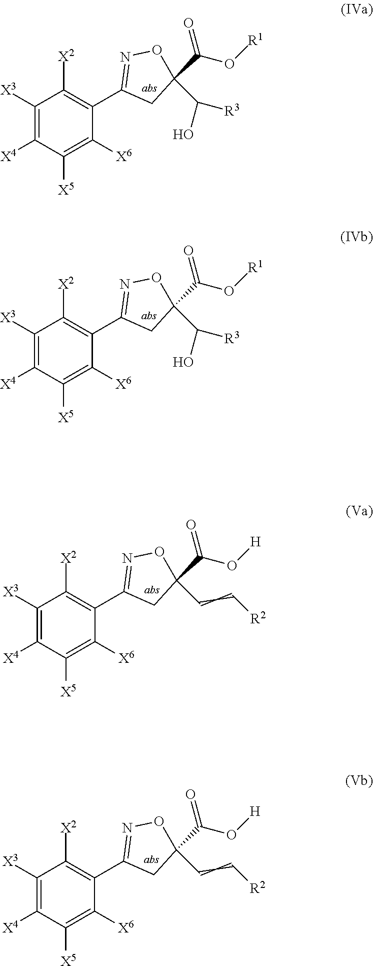

The terms used here are known to the person skilled in the art. Otherwise, the following definitions are used:

The C—C double bond

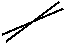

represents a cis or trans configuration of the respective radicals. This means that, for example, compounds of the formula (A)

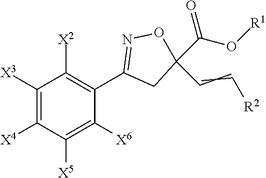

are understood to mean the configurations

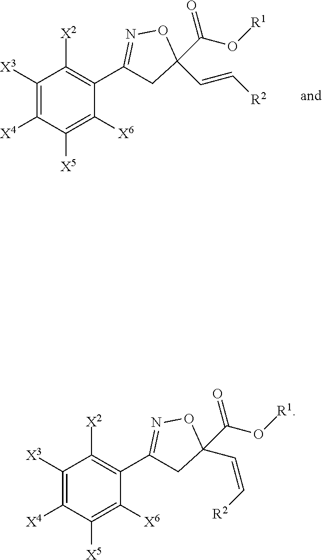

In the context of the present invention, unless defined differently elsewhere, the term “alkyl”, according to the invention, either on its own or else in combination with further terms, for example haloalkyl, is understood to mean a radical of a saturated, aliphatic hydrocarbon group which may be branched (isoalkyl, containing at least one secondary or tertiary or quaternary carbon atom in the alkyl chain) or unbranched (n-alkyl).

The term “alkoxy”, either on its own or else in combination with further terms, for example haloalkoxy, is understood in the present case to mean an O-alkyl radical, where the term “alkyl” is as defined above.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl.

Halogen-substituted radicals, for example fluoroalkyl, are mono- or polyhalogenated, up to the maximum number of possible substituents.

The ranges specified above generally or in preferred ranges apply correspondingly to the overall process. These definitions can be combined with one another as desired, i.e. including combinations between the respective preferred ranges.

Preference is given in accordance with the invention to using processes in which there is a combination of the meanings and ranges specified above as being preferred.

Particular preference is given in accordance with the invention to using processes in which there is a combination of the meanings and ranges specified above as being particularly preferred.

Very particular preference is given in accordance with the invention to using processes in which there is a combination of the meanings and ranges specified above as being very particularly preferred.

The greatest preference is given in accordance with the invention to using processes in which there is a combination of the meanings and ranges specified above as being most preferred.

### Elucidation of the Processes and Intermediates

### Step 0-1

The process according to the invention may comprise a step 0-1 in which the compounds of the formula (IV)

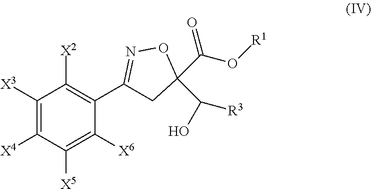

in which R³, R¹ and X² to X⁶ have the definitions given above are prepared by reacting compounds of formula (III)

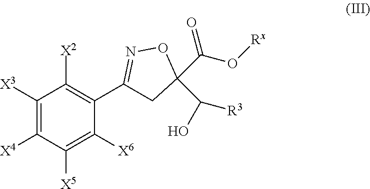

in which R³, R and X² to X⁶ have the definitions given above with compounds of the formula R¹—OH in which R¹ has the definition given above.

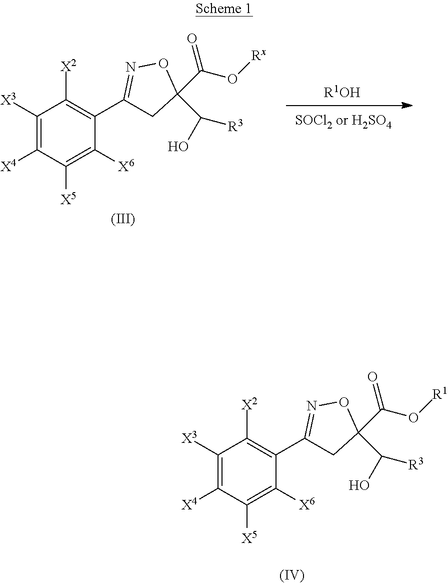

The preparation of compounds of the formula (III) is described, for example, in WO2018/228985.

The transesterification or esterification of the compound (III) with alcohols of the formula R¹—OH to give compound (IV) can be effected, for example, in the presence of 1.0 to 1.3 equivalents of thionyl chloride or catalytic amounts of sulfuric acid, based on the total molar amount of the compounds of the formula (III) used, at 0 to 80° C. (at standard pressure) for 1.5 to 3 h. Preference is given here to using the compounds of the formula R¹—OH as reactant and solvent in a distinct excess of, for example, 10 equivalents.

The transesterification or esterification of the compound (III) with alcohols of the formula R¹—OH to give compound (IV) can generally be conducted under any conditions known for such reactions in the prior art.

The compounds of the formula (IV) can be isolated by suitable workup steps generally known to those skilled in the art and characterized further, and can subsequently be used in step 0-2.

### Step 0-2

The process according to the invention may comprise a step 0-2 in which the compounds of the formula (II)

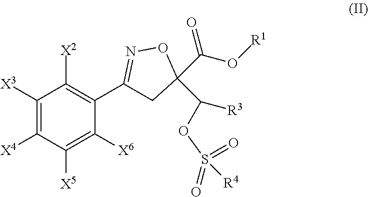

in which R¹, R³, R⁴ and X² to X⁶ have the meanings given above are prepared by reaction of compounds of the formula (IV)

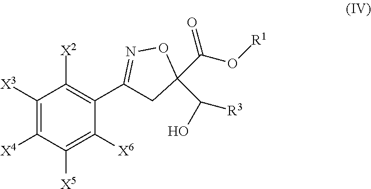

in which R¹, R³ and X² to X⁶ have the definitions given above
with R⁴SO₂Cl or (R⁴SO₂)₂O, where R⁴ has the meaning given above, in the presence of a base.

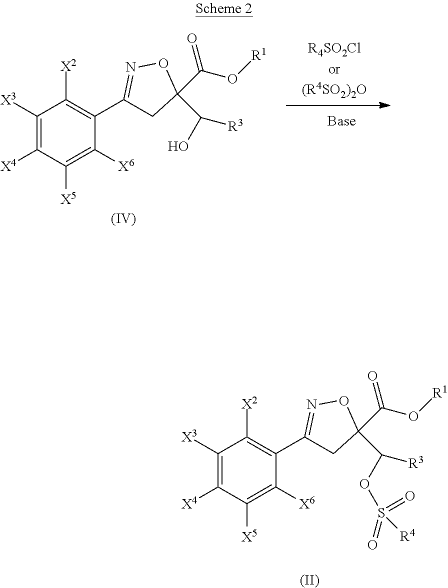

Preferably in accordance with the invention, suitable bases are selected from anhydrous organic bases, especially from triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide or N,N-dibutylformamide. Particular preference is given here to triethylamine, tripropylamine, tributylamine, N,N-diisopropylethylamine or N,N-dimethylcyclohexylamine.

According to the invention, suitable bases are also, albeit less preferably, anhydrous mixtures of organic and inorganic bases, for example the abovementioned organic bases with carbonates (for example (NH₄)₂CO₃, Li₂CO₃, Na₂CO₃, K₂CO₃, CaCO₃, MgCO₃).

The base is used here preferably in amounts between 1.0 and 5.0 equivalents, based on the total molar amount of the compounds of the formula (IV) used, more preferably between 1.05 and 3.0 equivalents, most preferably between 1.1 and 2.5 equivalents.

According to the invention, the compounds of the formula (IV) are reacted with R⁴SO₂Cl or (R⁴SO₂)₂O, where R⁴ has the definitions given above. R⁴ here is preferably C₁-C₄-alkyl or p-tolyl, more preferably C₁-C₂-alkyl or p-tolyl, even more preferably methyl or p-tolyl, and most preferably methyl. In an especially preferred configuration, methanesulfonyl chloride is used in step 1.

R⁴SO₂Cl or (R⁴SO₂)₂O is used here preferably in amounts between 1.0 and 5.0 equivalents, based on the total molar amount of the compounds of the formula (IV) used, more preferably between 1.05 and 3.0 equivalents, most preferably between 1.1 and 2.5 equivalents.

Step 0-2 is preferably conducted at an ambient temperature in the range from 0° C. to 50° C., more preferably in the range from 15° C. to 40° C. and most preferably in the range from 10° C. to 35° C. Cooling may be necessary for temperature compliance in the addition of R⁴SO₂Cl or (R⁴SO₂)₂O.

The reaction is preferably conducted in the region of standard pressure (1013 hPa), for example in the range from 300 hPa to 5000 hPa or from 500 hPa to 2000 hPa, preferably as in the range of 1013 hPa 200 hPa. Optionally, the reaction may alternatively be conducted under elevated or reduced pressure.

The reaction time for step 0-2 is preferably in the range from 0.5 h to 10 h, more preferably from 0.75 h to 5 h and most preferably 1 h to 4 h.

After the reaction, it is possible to remove any excess R⁴SO₂Cl or (R⁴SO₂)₂O by addition of alcohols, for example 2-propanol.

The compounds of the formula (II) can be isolated by suitable workup steps generally known to those skilled in the art, for example by extraction and optionally distillation, and characterized further, and can subsequently be used in step 1.

The reaction may be conducted in a solvent or solventlessly. Suitable solvents here are especially all the standard aprotic solvents, for example xylene, toluene, chlorobenzene, anisole, or the amines mentioned above as bases. The solvents may be used alone or in mixtures.

### Step 1

The process according to the invention comprises the preparation of isoxazoline-5,5-vinylcarboxylic acid derivatives of the formula (I)

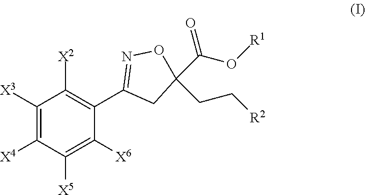

in which R¹, R² and X² to X⁶ have the definitions given above
by heating compounds of the general formula (II)

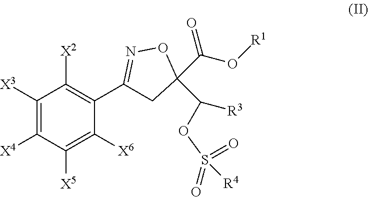

in which R¹, R³, R⁴ and X² to X⁶ have the meanings given above in the presence of a base to temperatures of 100 to 240° C.

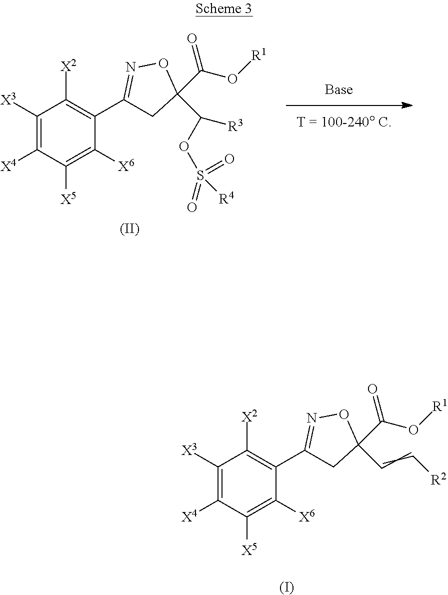

Preferably in accordance with the invention, suitable bases are selected from anhydrous organic bases, especially from triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide, N,N-dibutylformamide or alkoxy bases such as sodium methoxide, sodium tert-butoxide or sodium isopropoxide. Particular preference is given here to triethylamine, tripropylamine, tributylamine, N,N-diisopropylethylamine or N,N-dimethylcyclohexylamine.

According to the invention, suitable bases are also, albeit less preferably, anhydrous mixtures of organic and inorganic bases, for example the abovementioned organic bases with carbonates (for example (NH₄)₂CO₃, Li₂CO₃, Na₂CO₃, K₂CO₃, CaCO₃, MgCO₃).

The base is used here preferably in amounts between 1.0 and 10.0 equivalents, based on the total molar amount of the compounds of the formula (II) used, more preferably between 2.5 and 5.5 equivalents, most preferably between 3.0 and 5.0 equivalents.

Step 1 is preferably conducted at a temperature in the range from 100° C. to 240° C., more preferably in the range from 120° C. to 200° C. and most preferably in the range from 140° C. to 180° C.

The reaction is preferably conducted in the region of standard pressure (1013 hPa) or under elevated pressure, for example in the range from 300 hPa to 30 000 hPa, more preferably from 500 hPa to 6000 hPa.

The reaction time for step 1 is preferably in the range from 2 h to 60 h, more preferably from 3 h to 55 h and most preferably from 4 h to 50 h.

The reaction may be conducted in a solvent or solventlessly. Suitable solvents here are all the standard aprotic and protic solvents, for example xylene, toluene, chlorobenzene, anisole, isopropanol, 3-methyl-1-butanol, or the amines mentioned above as bases. The solvents may be used alone or in mixtures.

The compounds of the formula (I) can be isolated by suitable workup steps generally known to those skilled in the art, for example by extraction and optionally distillation, and characterized further, and can subsequently be used in step 2.

Preferably, the compounds of the formula (I) without further workup are hydrolysed in step 2 and only then isolated and purified.

### Step 2

The process according to the invention may further comprise the hydrolysis of compounds of the formula (I) to give compounds of the formula (V)

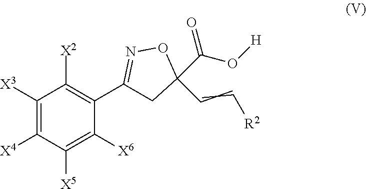

in which R² and X² to X⁶ have the definitions given above, in the presence of a base with subsequent protonation in the presence of an acid, or alternatively hydrolysis in the presence of an acid.

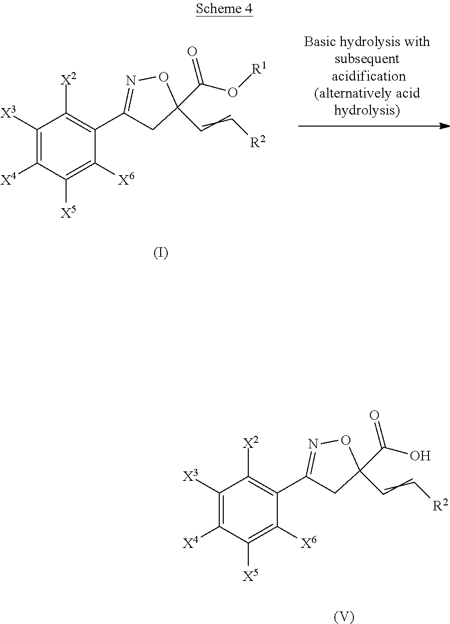

Suitable bases are especially inorganic bases, for example carbonates (e.g. (NH₄)₂CO₃, Li₂CO₃, Na₂CO₃, K₂CO₃, CaCO₃, MgCO₃), hydrogencarbonates (e.g. NH₄HCO₃, LiHCO₃, NaHCO₃, KHCO₃) or hydroxides (e.g. LiOH, NaOH, KOH, Ca(OH)₂); particular preference is given here to alkali metal or alkaline earth metal hydroxides, most preferably KOH or NaOH.

The base is preferably used in the form of an aqueous solution at concentrations of 1-50% by weight, more preferably of an aqueous solution at concentrations of 5-45% by weight, most preferably of an aqueous solution at concentrations of 5-35% by weight.

The reaction with base is preferably conducted at an ambient temperature in the range from 0° C. to 90° C., more preferably in the range from 10° C. to 80° C. and most preferably in the range from 15° C. to 60° C.

The reaction is preferably conducted in the region of standard pressure (1013 hPa), for example in the range from 300 hPa to 5000 hPa or from 500 hPa to 2000 hPa, preferably as in the range of 1013 hPa 200 hPa.

The reaction time for the hydrolysis is preferably in the range from 0.5 h to 10 h, more preferably from 0.75 h to 5 h and most preferably 1 h to 4 h.

The hydrolysis of the compounds of the formula (I) to give compound (V) can generally be conducted under any conditions known for such reactions in the prior art.

The compounds of the formula (I) can be isolated by suitable workup steps generally known to those skilled in the art, for example by extraction and optionally distillation, and characterized further.

In general, step 1 may be followed, rather than by the hydrolysis (step 2), also by a transesterification of the compound of the formula (I) at the R¹ position analogously to step 0-1.

Alternatively, step 2 may also be performed in the presence of an acid.

### Overall Process

In an advantageous configuration, the process according to the invention comprises steps 0-2 and 1, particularly advantageously 0-2, 1 and 2, and very particularly advantageously 0-1, 0-2, 1 and 2.

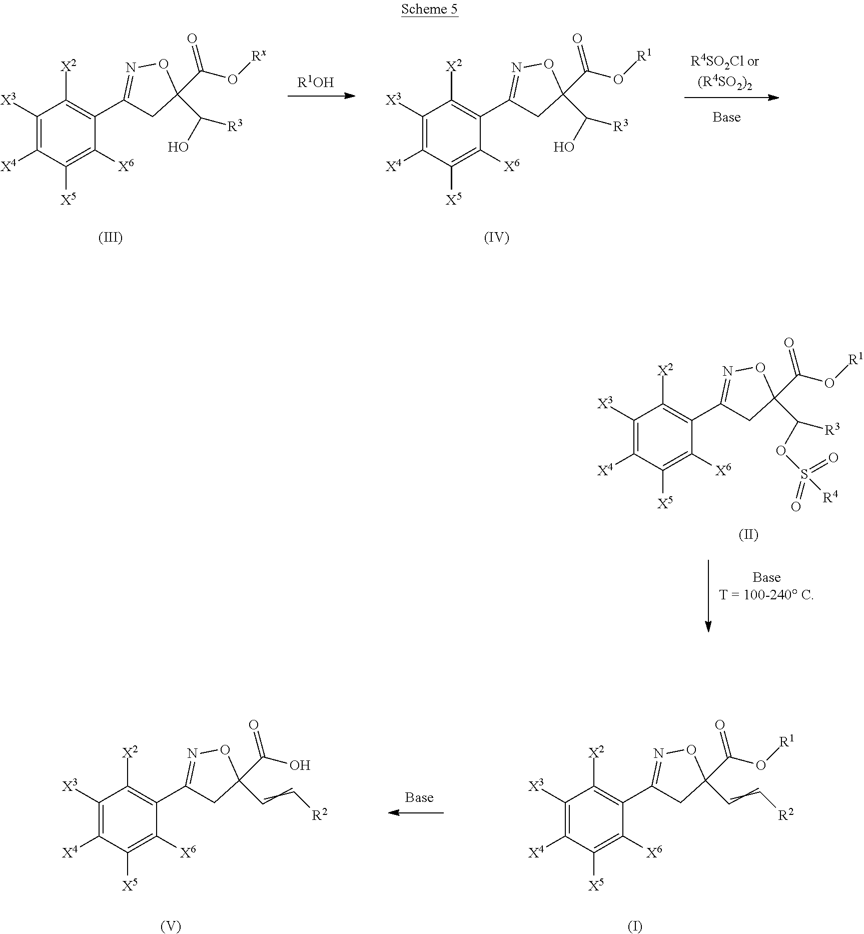

Scheme 5 gives a schematic overall representation of the process according to the invention, with all optional and obligatory steps. Reaction conditions and reactants are selected here in accordance with the above-described inventive and preferred configurations. All variables in the formulae are defined as described above.

The compounds of the formula (IV), (II) and (I) may be isolated and optionally also purified before being used in the respective next synthesis step. However, it is also possible that the compounds are used directly in the next step without isolation and purification. In this case, the solvent and excess reagents from the preceding stage are removed by standard methods before the compounds are used in the next synthesis step.

In a preferred configuration of the invention, the same base is used in steps 0-2 and 1.

### EXAMPLES

The present invention is elucidated in detail by the examples that follow, without restricting the invention thereto.

### Analysis Methods

The products were characterized by ¹H NMR and ¹⁹F NMR spectroscopy and/or HPLC (high-performance liquid chromatography).

The NMR spectra were determined using a Bruker Avance 400 fitted with a flow probe head (volume 60 μl). The NMR data of the examples are listed in conventional form (6 values, multiplet splitting, number of hydrogen or fluorine atoms).

The solvent and the frequency in which the NMR spectrum was recorded are stated in each case.

The HPLC (high-performance liquid chromatography) was conducted on an Agilent 1100 LC system with the following parameters: column: 100×4.6 mm, stainless steel; stationary phase: Daicel, Chiracel OZ-3; mobile phase: heptane/ethanol 90/10 (v/v), isocratic elution; oven temperature 40° C.; flow rate: 1.0 ml/min; run time 10 min, injection volume 5 μl. An instrument with UV detection and external standard quantification was used.

The yield of the respective products was determined by the following formula:

Rel. area % (single peak)=area (single peak)/total area of all peaks

### Step 0-1: Isopropyl 3-(3,5-difluorophenyl)-5-(1-hydroxyethyl)-4H-isoxazole-5-carboxylate

A suspension of 500 g of 3-(3,5-difluorophenyl)-5-(1-hydroxyethyl)-4H-isoxazole-5-carboxylic acid (1808 mmol, purity 98.1% by weight) in 1100 g of 2-propanol (99.0%) at 20° C. was heated to internal temperature 50° C. 260.1 g of thionyl chloride (2176 mmol, 99.5%) was added with a metering pump within 3 h. Subsequently, the solution was left to react further at 50° C. for a further 3 h. At the end of the reaction, a solid precipitated out of the solution, especially after suspension had been cooled to room temperature. The conversion of 3-(3,5-difluorophenyl)-5-(1-hydroxyethyl)-4H-isoxazole-5-carboxylic acid or the formation of isopropyl 3-(3,5-difluorophenyl)-5-(1-hydroxyethyl)-4H-isoxazole-5-carboxylate can be analysed by HPLC. The yield of the desired isopropyl 3-(3,5-difluorophenyl)-5-(1-hydroxyethyl)-4H-isoxazole-5-carboxylate was >98%. The reaction mixture was transferred into the next step without further treatment.

Step 0-2: Isopropyl 3-(3,5-difluorophenyl)-5-(1-methylsulfonyloxyethyl)-4H-isoxazole-5-carboxylate

The suspension formed in step 1 was heated to 60° C. Subsequently, 2-propanol was distilled off at 60-70° C. under reduced pressure. In the course of this, the pressure was reduced stepwise to 150 mbar. The distillation commenced at internal temperature 60° C. and at a pressure of about 450 mbar. The end point of the distillation was at about 70° C. and 150 mbar. Under these conditions, about 80% of the 2-propanol was distilled off before 1000 g of xylene was added stepwise and distillation was continued. The solution was cooled down to 60° C. and, at this temperature, 344 g of N,N-dimethylcyclohexylamine (99%, 2677 mmol) was added to the mixture. The resultant solution was cooled down to 20° C. Subsequently, 272 g of methanesulfonyl chloride (2351 mmol, 99%) was added at 20-25° C. within about 2 h. Once the addition of methanesulfonyl chloride had concluded, the reaction mixture was left to react further at 20° C. for another 1 hour. 150 g of water and 500 g of K₂CO₃ solution (25% by weight) were added at 20° C. After phase separation at 40° C., 1128 g of water phase was released to wastewater treatment, and 1767 g of xylene phase (40.1% by weight of isopropyl 3-(3,5-difluorophenyl)-5-(1-methylsulfonyloxyethyl)-4H-isoxazole-5-carboxylate in xylene solution) was isolated. The yield of the desired isopropyl 3-(3,5-difluorophenyl)-5-(1-methylsulfonyloxyethyl)-4H-isoxazole-5-carboxylate in xylene was found by the standard HPLC method to be >98%.

Step 1: Isopropyl 3-(3,5-difluorophenyl)-5-vinyl-4H-isoxazole-5-carboxylate

1767 g of solution of isopropyl 3-(3,5-difluorophenyl)-5-(1-methylsulfonyloxyethyl)-4H-isoxazole-5-carboxylate (1806 mmol, purity 40.1% by weight) in xylene from step 2 was heated to internal temperature 80° C. Subsequently, the xylene was distilled off at 78 to 107° C. under reduced pressure. In the course of this, the pressure was reduced stepwise to 12 mbar. The end point of the distillation was at about 108° C. and 12 mbar. 975 g of N,N-dimethylcyclohexylamine (7663 mmol, 99%) was added at 99° C. and 1013 mbar to the residual melt of isopropyl 3-(3,5-difluorophenyl)-5-(1-methylsulfonyloxyethyl)-4H-isoxazole-5-carboxylate. 80 g was subsequently distilled off at 92° C. and 90 mbar in order to remove the residual xylene. The solution was heated to jacket temperature 155° C. and internal temperature about 149° C. for 52 hours. The conversion of isopropyl 3-(3,5-difluorophenyl)-5-(1-methylsulfonyloxyethyl)-4H-isoxazole-5-carboxylate or the formation of isopropyl 3-(3,5-difluorophenyl)-5-vinyl-4H-isoxazole-5-carboxylate can be analysed by HPLC. The isopropyl 3-(3,5-difluorophenyl)-5-(1-methylsulfonyloxyethyl)-4H-isoxazole-5-carboxylate content was <1%.

The yield of isopropyl 3-(3,5-difluorophenyl)-5-vinyl-4H-isoxazole-5-carboxylate was only determined indirectly using the yield after the hydrolysis (step 2).

Step 2: 3-(3,5-Difluorophenyl)-5-vinyl-4H-isoxazole-5-carboxylic acid

The reaction mixture from step 3 was cooled to about 30° C. Subsequently, 607 g of water was added, followed by the addition of 453 g of sodium hydroxide solution (32% by weight), within 2 hours at a temperature of 25 to 30° C. This was followed by stirring at 30° C. for a further hour. The hydrolysis was analysed by HPLC. On completion of hydrolysis, the mixture was distilled at 50-57° C. under reduced pressure, in order to remove N,N-dimethylcyclohexylamine by azeotropic distillation; the lower water phase was returned to the reaction mixture. 200 g of xylene was added to the distillation bottoms at 25° C. After the phase separation at 40° C., 199 g of organic phase was released to wastewater treatment, and 1428 g of aqueous phase was isolated. 1004 g of xylene and 428 g of hydrochloric acid (20% by weight) were added to the aqueous phase at 20° C. After phase separation at 20° C., 1215 g of water phase was released to wastewater treatment, and 1508 g of xylene phase (23.5% by weight of 3-(3,5-difluorophenyl)-5-vinyl-4H-isoxazole-5-carboxylic acid in xylene) was isolated. The yield of the desired 3-(3,5-difluorophenyl)-5-vinyl-4H-isoxazole-5-carboxylic acid in xylene was determined by HPLC and was 77.5%.

The NMR data of the isolated and purified products and intermediates were determined as follows:

### Isopropyl 3-(3,5-difluorophenyl)-5-(1-hydroxyethyl)-4H-isoxazole-5-carboxylate (after step 0-1)

¹H-NMR (400 MHz, CDCl₃): δ(ppm)=1.28-1.32 (m, 9H), 2,18 (s, 1H), 3.53 (d, J=17.4 Hz, 1H), 3.67 (d, J=17.4 Hz, 1H), 4.22 (q, J=6.5 Hz, 1H), 5.13 (hept, J=6.3 Hz, 1H), 6.84-6.91 (m, 1H), 7.15-7.22 (m, 2H).

¹⁹F-NMR (376 MHz, CDCl₃): δ(ppm)=−108.4 (m, 2F).

### Isopropyl 3-(3,5-difluorophenyl)-5-(1-methylsulfonyloxyethyl)-4H-isoxazole-5-carboxylate (after step 0-2)

¹H-NMR (400 MHz, CDCl₃): δ(ppm)=1.33 (pst, J=6.3 Hz, 6H), 1.52 (d, J=6.5 Hz, 3H), 3.05 (s, 3H), 3.59 (d, J=17.7 Hz, 1H), 3.72 (d, J=17.7 Hz, 1H), 5.14 (hept, J=6.3 Hz, 1H), 5.32 (q, J=6.5 Hz, 1H), 6.86-6.92 (m, 1H), 7.15-7.23 (m, 2H).

¹⁹F-NMR (376 MHz, CDCl₃): δ(ppm)=−108.1 (m, 2F).

### Isopropyl 3-(3,5-difluorophenyl)-5-vinyl-4H-isoxazole-5-carboxylate (after step 1)

¹H-NMR (401 MHz, CDCl₃): δ(ppm)=1.31 (dd, J=6.3, 1.0 Hz, 6H), 3.31 (d, J=17.0 Hz, 1H), 3.89 (d, J=17.0 Hz, 1H), 5.11 (hept, J=6.3 Hz, 1H), 5.36 (d, J=10.7 Hz, 1H), 5.54 (d, J=17.2 Hz, 1H), 6.13 (dd, J=17.2, 10.7 Hz, 1H), 6.84-6.90 (m, 1H), 7.15-7.22 (m, 2H).

¹⁹F-NMR (376 MHz, CDCl₃): δ(ppm)=−108.4 (m, 2F).

### 3-(3,5-Difluorophenyl)-5-vinyl-4H-isoxazole-5-carboxylic acid (after step 2)

¹H-NMR (400 MHz, CDCl₃): δ(ppm)=3.40 (d, J=17.1 Hz, 1H), 3.92 (d, J=17.1 Hz, 1H), 5.44 (d, J=10.7 Hz, 1H), 5.63 (d, J=17.2 Hz, 1H), 6.16 (dd, J=17.2, 10.7 Hz, 1H), 6.86-6.92 (m, 1H), 7.14-7.21 (m, 2H), 9.61 (bs, 1H).

¹⁹F-NMR (376 MHz, CDCl₃): δ(ppm)=−108.0 (m, 2F).

Analogously to the above-described procedure for step 1, further compounds of the formula (I) were prepared.

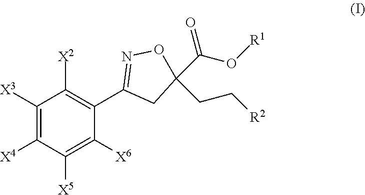

All variables in the compounds of the formula (I) and (II), except for R¹, were chosen as in the above example.

Table 1 summarizes the experiments and states the parameters that differ from the above procedure.

The yields were determined by HPLC as specified above.

TABLE 1

| | | | | | | Yield of |
|---|---|---|---|---|---|---|
| | | | | | Yield of | compound |
| | | | | | compound | of formula |
| | | | Reaction | | of formula | (IV)**, |
| | Base | Temperature | time | Conversion | (I), HPLC | HPLC |
| R¹ | (equivalents) | (° C.) | (h)* | (%) | area % | area % |
| isopropyl | N,N-Dimethylcyclohexylamine | 155-160 | 20.5 | 98 | 85 | 86 |
| | (3) | | | | | |
| isopropyl | N,N-Dimethylcyclohexylamine | 155-160 | 20.5 | 98 | 87 | 84 |
| | (5) | | | | | |
| 2-methylpropan-1-yl | N,N-Dimethylcyclohexylamine | 160 | 17 | 99 | 86 | not |
| | (5) | | | | | determined |
| n-butyl | N,N-Dimethylcyclohexylamine | 160 | 14 | 98 | 83 | not |
| | (5) | | | | | determined |
| cyclohexyl | N,N-Dimethylcyclohexylamine | 155 | 19 | 99 | 90 | not |
| | (5) | | | | | determined |
| propyl | N,N-Dimethylcyclohexylamine | 152 | 13 | 98 | 75 | not |
| | (5) | | | | | determined |
| ethyl | N,N-Dimethylcyclohexylamine | 160 | 7 | 98 | 58 | not |
| (comparative) | (5) | | | | | determined |
| methyl | N,N-Dimethylcyclohexylamine | 130-138 | 1.33 | 99 | 1.7 | not |
| (comparative) | (5) | | | | | determined |

*The reaction was ended when virtually complete conversion of the reactant compound of the formula (II) was observed by HPLC.
**after hydrolysis according to step 2 described by way of example above

It is apparent from Table 1 that the resultant yield of compounds of the formula (I) is highly dependent on the selection of variable R¹.

1. A process for preparing isoxazoline-5,5-vinylcarboxylic acid derivatives of formula (I) 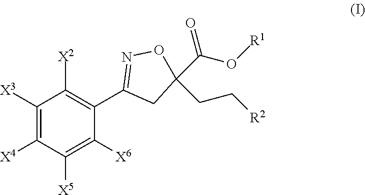
in which
X² is H, C₁-C₄-alkyl, C₁-C₄-fluoroalkyl, C₁-C₄-fluoroalkoxy, C₁-C₄-alkoxy, fluorine or CN,
X³ is H, C₁-C₄ alkyl, C₁-C₄ fluoroalkyl, C₁-C₄ fluoroalkoxy, C₁-C₄ alkoxy, fluorine, chlorine or CN,
X⁴ is H, C₁-C₄-alkyl, C₁-C₄-fluoroalkyl, C₁-C₄-fluoroalkoxy, C₁-C₄-alkoxy, fluorine or CN,
X⁵ is H, C₁-C₄ alkyl, C₁-C₄ fluoroalkyl, C₁-C₄ fluoroalkoxy, C₁-C₄ alkoxy, fluorine, chlorine or CN,
X⁶ is H, C₁-C₄-alkyl, C₁-C₄-fluoroalkyl, C₁-C₄-fluoroalkoxy, C₁-C₄-alkoxy, fluorine or CN,
R¹ is branched C₃-C₈-alkyl, n-C₃-C₈-alkyl, C₃-C₈-cycloalkyl, unsubstituted benzyl, unsubstituted phenyl or mono- or di-C₁-C₃-alkyl-substituted benzyl or phenyl and
R² is H or C₁-C₃-alkyl,
the process comprising heating, to a temperature of between 10° and 240° C. in the presence of a base, at least one compound of general formula (II) 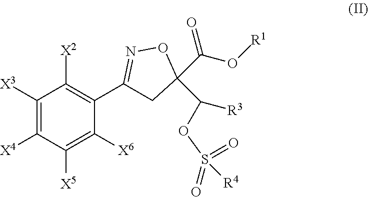
in which R¹ and X² to X⁶ have the meanings given above,
R³ is C₁-C₄-alkyl and
R⁴ is C₁-C₄-alkyl, unsubstituted phenyl or mono- or di-C₁-C₃-alkyl-substituted phenyl.

2. The process according to claim 1, wherein the base is selected from the group consisting of triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide, and N,N-dibutylformamide.

3. The process according to claim 1, wherein the temperature is between 120° C. and 200° C.

4. The process according to claim 1, further comprising preparation of at least one compound of formula (II) 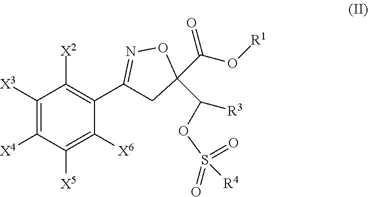
by a reaction of compounds of formula (IV) 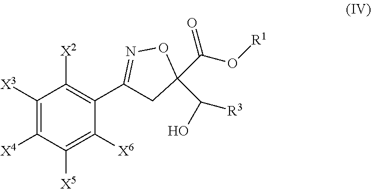
in which R¹, R³, R⁴ and X² to X⁶ have the meanings given in claim 1
with R⁴SO₂Cl or (R⁴SO₂)₂O, where R⁴ has the meaning given in claim 1,
in the presence of a base.

5. The process according to claim 4, wherein the reaction is conducted at a temperature of between 0 and 50° C.

6. The process according to claim 4, wherein the base is selected from triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide and N,N-dibutylformamide.

7. The process according to claim 1, further comprising preparation of at least one compound of formula (IV) by a reaction of at least one compound of formula (III) 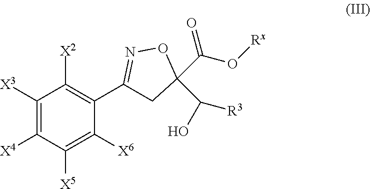
in which
R³ and X² to X⁶ have the definitions given in claim 1 and
R^{x} is H or C₁-C₃-n-alkyl, where, if R¹ is n-propyl, R^{x} is not n-propyl, with compounds of formula R¹—OH in which R¹ has the definition given in claim 1.

8. The process according to claim 1, further comprising a reaction of at least one compound of formula (I) 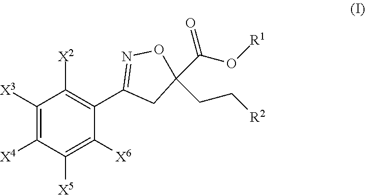
in which R¹, R² and X² to X⁶ have the definitions given in claim 1, wherein the reaction is carried out in the presence of a base to give compounds of formula (V) 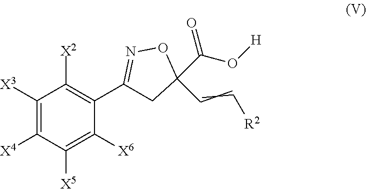
in which R² and X² to X⁶ have the definitions given in claim 1.

9. The process according to claim 8, wherein the base is an inorganic base.

10. The process according to claim 1, wherein
X² is H,
X³ is H or fluorine,
X⁴ is H or fluorine,
X⁵ is H or fluorine,
X⁶ is H.

11. The process according to claim 1, wherein R¹ is isopropyl, n-propyl, 2-methyl-1-propyl, 1-methyl-1-propyl, cyclohexyl, 3-methyl-1-butyl, 1-butyl, 1-pentyl, benzyl or tert-butyl.

12. The process according to claim 1, wherein R⁴ is one of a C₁-C₄-alkyl, p-tolyl, and methyl.

13. At least one compound according to formula (I) 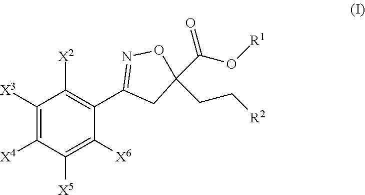
in which R¹, R² and X² to X⁶ have the definitions given in claim 1.

14. At least one compound according to formula (II) 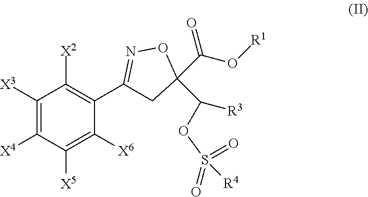
in which R¹, R³, R⁴ and X² to X⁶ have the definitions given in claim 1.

15. At least one compound according to formula (IV) 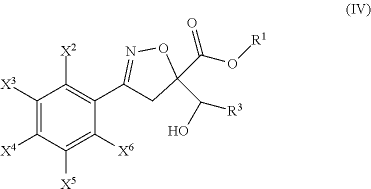
in which R¹, R³ and X² to X⁶ have the definitions given in claim 1.

16. The process according to claim 1, wherein the base is an alkoxy base.

17. The process according to claim 16, wherein the alkoxy base is selected from sodium methoxide, sodium tert-butoxide, and sodium isopropoxide,

18. The process according to claim 2, wherein the base is selected from triethylamine, tripropylamine, tributylamine, N,N-diisopropylethylamine and N,N-dimethylcyclohexylamine.

19. The process according to claim 6, wherein the base is selected from triethylamine, tripropylamine, tributylamine, N,N-diisopropylethylamine and N,N-dimethylcyclohexylamine.

20. The process according to claim 9, wherein the inorganic base is selected from alkali metal and alkaline earth metal hydroxides.

21. The process according to claim 12, wherein R⁴ is methyl.
